(19) United States
(12) Patent Application Publication
(10) Pub. No.: US 2007/0157935 A1
(43) Pub. Date: Jul. 12, 2007

(54) Medical emergency equipment

(76) Inventor: Connell Reynolds, Fairburn, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/328,041

(22) Filed: Jan. 9, 2006

(51) Int. Cl. A61B 19/00

(52) U.S. Cl. 128/869

(57) ABSTRACT

A medical emergency head and neck stabilization system for use on a patient carrying device such as a spine board includes a sleeve of cushioning material which fits over the end of the spine board. First and second side head restraints are affixable to the cushioning material and a head retaining strap is designed to pass over the patient's head, such as the forehead, and to be fixed in position on or around the sleeve.

### FIELD OF THE INVENTION

The present invention relates generally to medical emergency equipment and, more particularly, to a disposable patient head and neck immobilization assembly usable in extremes of weather as well as normally.

### BACKGROUND OF THE INVENTION

In medical emergencies and accidents, it is often necessary to immobilize a victim or patient prior to transporting the patient to a medical facility. Typically, a patient is placed on an immobilization board, known as a spine board, which maintains the patient in a flat, straight position. Additionally, in order to prevent further injury, the patient's head and neck must be further immobilized, typically by securing the patient's head to the spine board. Prior art devices for accomplishing such immobilizations are complex in design, difficult to apply and remove, and costly to manufacture. In most injury cases, one of the most important steps to be taken is the quick and safe immobilization of the patient. If immobilization requires manipulation of the patient's head and/or neck, then the goal of quick and safe immobilization is not realized. For example, the cervical collar disclosed in U.S. Pat. No. 4,043,325, which wraps around a patient's neck, requires that the patient's head be manipulated in order to secure the collar around the patient's neck which can cause further, often irreparable injury in some case. Similarly, the emergency neck immobilizer disclosed in U.S. Pat. No. 4,732,144 requires that the patient be manipulated in order to apply the device, which is complex in design and costly to manufacture. While these devices may help to immobilize a patient, they are usually cumbersome and uncomfortable, and may add to the feeling of anxiety being experienced by the patient, by being overly restrictive as well as posing a risk to the patient by manipulation of his or her head or neck. Furthermore, these prior art devices are not designed to be disposable, and therefore, must be sterilized after each use. This presents a hazard of contamination from various bodily fluids and presents an increased health risk.

In U.S. Pat. Nos. 5,967,144 and 5,785,058, both issued to the present inventor, there are shown disposable head and neck immobilization devices which comprise a mask device of rugged denier nylon for covering the patient's head, and each one has a large opening therein for allowing the patient to breath easily and to see clearly. Such an opening does much to alleviate any anxiety and discomfort that the patient may feel. The mask is strapped, or otherwise mounted to, the spine board and functions to prevent movement in any direction of the patient's head and neck. Application of these masks requires a minimum of head manipulation.

Most of the prior art arrangements present certain problems in inclement weather. Those that are made of nylon, for example, can become soaked, greatly increasing the discomfort of the patient and, in some cases, diminishing the effectiveness of the apparatus. Those that are made of plastic are substantially immune to atmospheric effects, but are generally uncomfortable for the patient to begin with. It is often necessary or desirable to place a cushion under the patient's head but the cushion itself can become soaked with the concomitant increase in patient discomfort. Also, a cushion under the head can result in the head being tilted which can cause a slight blockage of the patient's air passage resulting in some breathing impairment, which, for certain types of injuries, can be critical.

### SUMMARY OF THE INVENTION

The present invention is a disposable head and neck immobilization assembly that is, in its function and reliability, substantially impervious to the effects of weather or other ambient effects, and requires a minimum of patient manipulation, as well as maintaining clear breathing passages while allowing the patient to see and hear what is happening.

In a preferred embodiment of the invention, the assembly comprises a sleeve of cushioning material such as preferred plastic bubble wrap which is dimensioned to be slipped over the spine board at the head end thereof. The top surface of the sleeve is preferably smooth, and the interior surface of the sleeve contains the air bubbles. The top surface of the sleeve has one or two large areas of adhesion material, for example, patches of Velcro® loops which are located on either side of the top surface of the sleeve centerline or extend across the top surface of the sleeve and are cemented in place. First and second head restraining blocks of, for example, Styrofoam, have a bottom surface with Velcro® hooks for firmly attaching the blocks to the large area surfaces. The blocks are, in use, spaced to bear against the sides of the patient's head to immobilize it from side-to-side or lateral movement without the necessity of moving the patient's head. The movable blocks can easily be positioned to accommodate the lateral dimensions of the patient's head. When the patient is lying on the spine board, his head is cushioned by the bubble wrap material between the two head restraint blocks. The bubble wrap is preferable to a pillow or other cushioning device in that it does not raise the patient's head and therefore it affords cushioning without blocking the patient's airway, which, as pointed out, often results from a raised position of the head.

The patient's head is further immobilized by a single strap extending from and affixed to one side of the spine board bubble wrap sleeve, as by Velcro®, or other adhesion type material to the other side over the patient's forehead. The undersurface portion of the strap preferably has a light adhesive layer or pad thereon which lightly adheres to the patient's forehead to prevent slippages and to prevent vertical and rotational movement of the patient's head. In addition, it is desirable that the patient's body be strapped to the spine board to prevent bodily movement from possibly causing movement of the patient's head.

When the patient is thus immobilized, being strapped to the spine board and with the head and neck immobilized by the restraint assembly of the invention, the patient's face is readily accessible, his nose and mouth are clear for easy breathing, the eyes are clear of any encumbrances or obstruction, and the patient's relative comfort and diminished anxiety is assured. Further, the restraint assembly of the invention is relatively immune from the detrimental effects of inclement weather, such as rain, especially due to Velcro® fastenings. The entire restraint assembly is designed for single use, and may be discarded when no longer needed. As a consequence, cleaning and sterilization are unnecessary.

Spine boards may come in a variety of widths, such as, for example, fourteen inches to twenty inches. In another embodiment of the invention, the sleeve is made of a top layer of bubble wrap and a bottom layer, which are joined at their longitudinal edges by elastic material panels which enable the sleeve to fit snugly on virtually any width of spine board.

The invention has numerous advantages, principal among which are a quick immobilization of the patient, primarily his or her head with virtually no manipulation of the patient's head or neck, and while maintaining communication between the patient and emergency personnel. Additionally, the patient's sight, hearing and breathing are virtually unimpaired.

The apparatus of the invention is both simple in form and in use, and is made of materials that are readily available and not costly, which allows disposal of the apparatus after one use, a very important sanitary feature.

In addition to the foregoing advantages, in use, the application of the restraint assembly to the spine board and the patient is quickly accomplished, compared to prior art arrangements, which is an extremely desirable feature, inasmuch as speed is usually of the essence, and manipulation of the patient is minimized.

These and other features and advantages of the present invention will be readily apparent from the following detailed description, read in conjunction with the accompanying drawings.

### BRIEF DESCRIPTION OF THE DRAWINGS

FIG. 1 is an exploded perspective view of the various components of the apparatus of the invention;

FIG. 2a is a plan view of one embodiment of the sleeve of the invention;

FIG. 2b is an end view of the sleeve of FIG. 2a;

FIG. 3a is a plan view of a second embodiment of the invention;

FIG. 3b is an end view of the sleeve of FIG. 3a;

FIG. 4 is a detail of the sleeve depicted in FIG. 3b;

FIG. 5 is a plan view of the underside of the immobilizing strap of the invention.

FIG. 6a is a side elevation view of the head restraining block of the invention;

FIG. 6b is a bottom view of the head restraining block of FIG. 5a; and

FIG. 6c is a front elevation view of the head restraining block of FIG. 5a.

### DETAILED DESCRIPTION

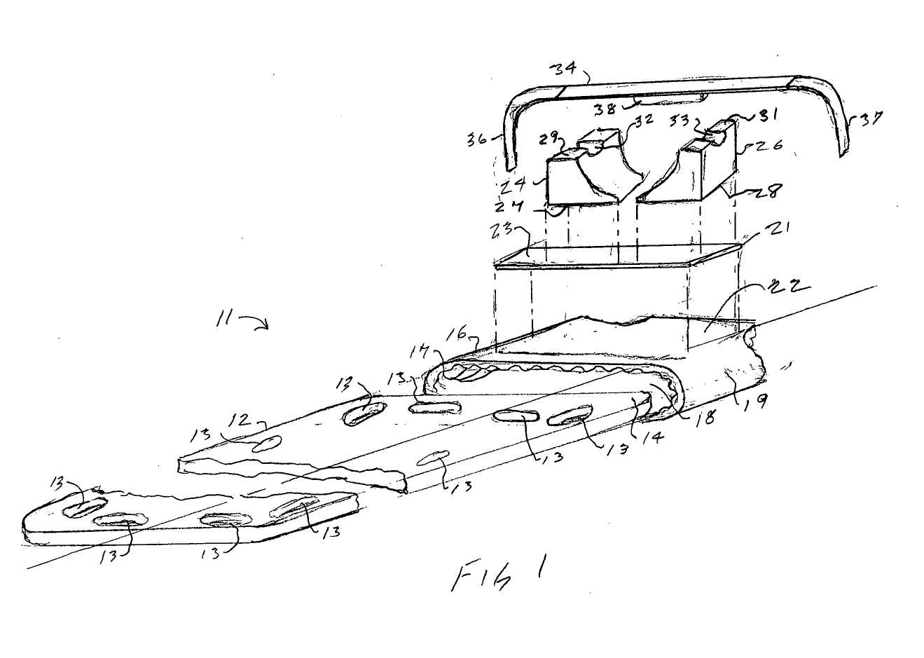

FIG. 1 is an exploded perspective view of a first embodiment 11 of the present invention for use with emergency equipment. Common to all such emergency equipment is a spine board 12 which is standard equipment in paramedic and rescue vehicles. Board 12 may be made of any of a number of rigid light-weight material such as, for example, a hard plastic. A plurality of elongated holes 13 are strategically placed for functioning as hand holds for lifting and carrying the spine board. Holes 13 may also function as passages for straps and the like, not shown, especially for use in strapping the patient to the board.

The various elements which constitute the apparatus of the invention are adapted to fit upon the head end 14 of the spine board 12. Thus the apparatus of the invention comprises a sleeve 16 of commercially available bubble wrap formed so that the plastic bubbles 17 are on the interior 18 of the sleeve, and its exterior surfaces 19 are smooth. Sleeve 16 is adapted to being slid onto the spine board and to fit snugly thereon. Inasmuch as spine boards 12 are made in differing widths, from fourteen inches to twenty inches, for example, the interior width 18 of the sleeve 16 is dimensioned in this embodiment for one particular width spine board 12. This does not necessarily represent an inconvenience inasmuch as most emergency vehicles carry only one particular width of spine board, hence they are to be equipped with sleeves 16 of the desired width.

A Velcro® or other fastening type material patch or strip 21 is cemented to the smooth top surface 22 of the sleeve 16. Velcro® fasteners comprise one member having a surface of material having a multitude of minute loops, and a second material having a surface of a multitude of minute hooks, which are adapted to mate with the loops. The top surface 23 of patch 21 is preferably although not necessarily, the loop portion of a Velcro® fastening assembly inasmuch as it is softer and more resilient than the hook portion, and, as will be discussed hereinafter, the back of the patient's head rests upon it.

The patch 21 may comprise two spaced Velcro® patches so that the patient's head may rest upon the upper surface 22 of the sleeve between the two patches. One of the advantages of Velcro® is that the two members may be attached by pressing them together and then detached by simply pulling them apart, and the fastening assembly is reusable as needed.

First and second substantially identical head restraint members 24 and 26 are adapted to be placed on the pad 21 on either side of the patient's head to prevent it from moving during transport. The bottom surface of each of the restraint members has a Velcro® hook pad 27, 28 cemented so that when placed on either side of the patient's head, the restraint members 24 and 26 are firmly held in position by the mating of the pad surface 23 with the hook pads 27 and 28. The top surfaces 29 and 31 of the restraint members 24 and 26 have, respectively, notches 32 and 33, the purpose of which will be clear hereinafter. Members 24 and 26 are preferably made from a very dense polyurethane based foam plastic of polyethylene or polypropylene, for example, yet is soft enough to accommodate the patient's head comfortably. Further, to prevent the foamed material from absorbing moisture, the members 24 and 26 preferably have a skin formed thereon which is substantially moisture proof.

As thus far described, the assembly 11 provides for immobilizing the patient by strapping him or her to the spine board, with his or her head resting on the relatively soft surface 23 of pad 21 and cushioned by the sleeve 16. The patient's head lies in a substantially flat position, with no bending of the neck, thus minimizing any blockage of his or her breathing passages. The restraint members 24 and 26 hold the head against lateral movement for protection against possible injury resulting from such movement.

It is possible, even with the assembly as thus far described, that the patient may attempt to rotate his or her head, or that it may accidentally be so rotated. In addition, the head may be raised by the patient or by accident, such as the emergency vehicle passing over bumpy surfaces. To guard against such movement of the head, a retaining strap 34 is provided. Strap 34 is preferably made of a flexible plastic material and has, at each end, matching Velcro® patches 36 and 37. In the present embodiment of the invention, one patch, such as patch 36 comprises loop material, and the other patch 37 comprises hook material. Strap 34 is long enough to pass over the patient's forehead, through notches 32 and 33, around the edges of sleeve 16 and patches 36 and 37 mated on the underside of sleeve 16 of the spine board 12. Centrally located on the underside of strap 34 is a cushioning pad 38 which, when the strap is properly in place, bears against the patient's forehead, for comfort. Pad 38 may have a lightly adhesive material on its surface to hold it in place, against the patient's forehead. As a consequence, the patient's head is substantially totally immobilized. The immobilization arrangement of the present embodiment of the invention is easy to apply and remove with virtually no manipulation of the patient and produces a decreased level of anxiety by allowing the patient to see and hear what is happening around them, and allows the patient, if able, to communicate freely. In addition, the apparatus of the invention may be discarded after a single use, thus eliminating the necessity of sterilization and the possibility of contamination.

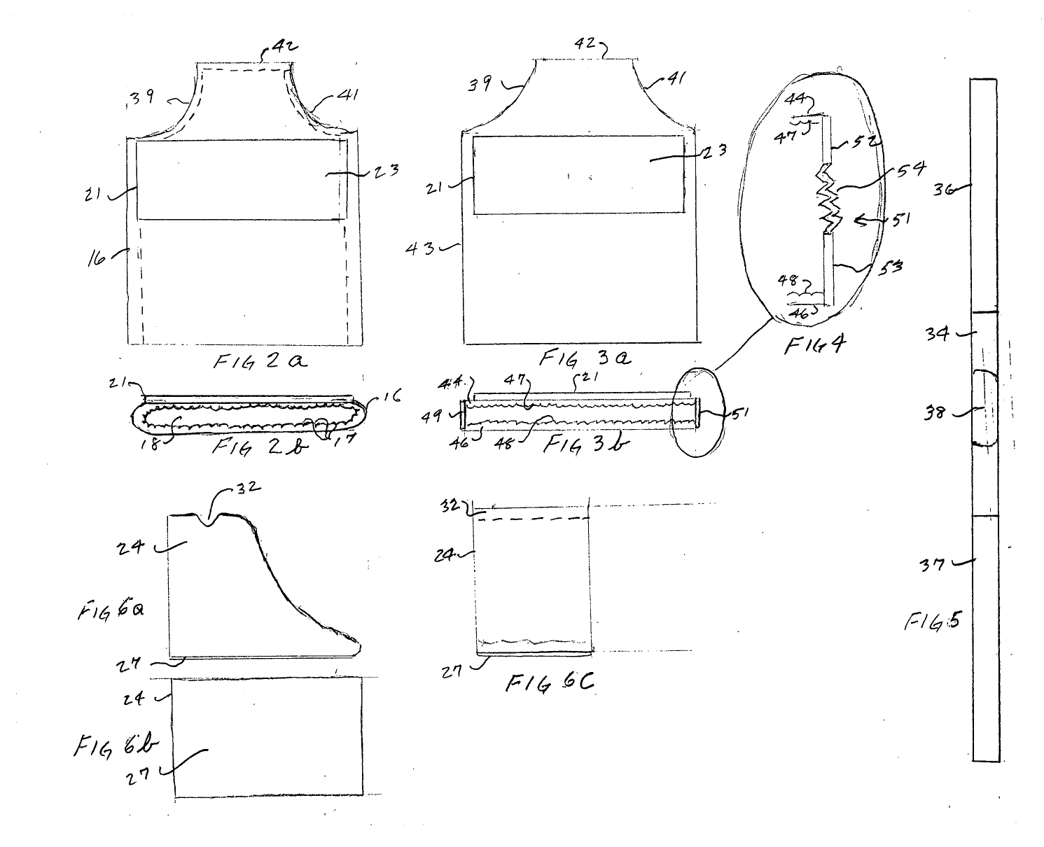

FIGS. 2a and 2b are, respectively, a plan view and a front elevation view of the sleeve 16 of the first embodiment of the invention. In order that the hand holds 13 at the head end 14 of the spine board 12 be made accessible, sleeve 16 has large cut-out portions 39 and 41, extending as shown from the top or head or closed end 42 of sleeve 16 to the sides thereof. The cut-out portions 39 and 41 do not, in any way, impair the function of the assembly of the invention.

FIGS. 3a and 3b depict a second embodiment of the invention in, respectively, a plan view and a front elevation view of a universal sleeve 43 which is readily usable with virtually any width spine board. In the figures the same reference numerals identify like parts with those of FIGS. 1, 2a, and 2b. Sleeve 43 comprises an upper member 44 of bubble wrap material and a lower member 46, with the top surface of member 44 being smooth and the bubble surface 47 thereof facing the bubble surface 48 of member 46, as shown in FIG. 3b. Members 44 and 46 are joined at their substantially parallel side ends by an elastic material 49 and 51 as shown in FIG. 3b, the details of which are shown in FIG. 4.

As can be seen in FIG. 4, strip 51 (and strip 49) comprises a first strip 52 of Velcro®, for example, which is cemented to the side edge of bubble wrap member 44, as shown, and a second strip 53 of Velcro® material is cemented to the side edge of member 46. The material of the strip 52 and 53 are preferably the hook portion of a Velcro® attachment, but the material may be the loop portion. Strips 52 and 53 are joined together by a strip of expansible or elastic material 54 which may be pleated, as shown, and which is preferably sewn thereto for strength, although it may be cemented, provided the cement forms a strong enough bond. The sleeve 43 thus allows an expansion or contraction of the width of the sleeve for use with spine boards of differing widths, thus it is not necessary for the emergency vehicle to be equipped with sleeves of varying widths.

FIG. 5 is a plan view of the underside of the strap 34 having, at each end thereof strips 36 and 37 of Velcro®, which mate with each other when the embodiment of FIG. 1 is used. In use, the cushioning pad 38 is placed against the patient's forehead and strap 34 passes over the head restraint member 24 and 26 through notches 32 and 33 and the ends 36 and 37 are joined on the underside of the sleeve 16. Notches 32 and 33 prevent the strap 34 from slipping when placed over the patient's head. Pad 38 protects the patient's head from possible irritation by strap 34, and may be equipped with a mildly adhesive surface to hold strap 34 in place on the patient's head. In the embodiment of FIGS. 3a, 3b, and 4, the fastening portion 36 and 37 are intended to mate with portions 52 and 53 of side members 44 and 46, hence if portions 52 and 53 are Velcro® loop members, portions 36 and 37 are hook members. This arrangement permits tightening and hence securing the sleeve 16 to the spine board 12.

FIGS. 6a, 6b, and 6c are, respectively, a side elevation view of head restraint member 24 (member 26 being substantially identical thereto), a bottom view of member 24, and a front elevation view thereof. as discussed in the foregoing, the bottom surface of member 24 has a Velcro® pad 27 cemented thereon. In the preferred configuration, pad 27 is the hook portion of a Velcro® attachment and pad 23, which mates with pad 27, is the loop portion, as previously discussed, all the opposite configuration might be used. The depth of notch 32 should be sufficient to prevent sideways slipping of strap 34 when it is positioned in the notch 32. The notch 32 can be made deep enough so that the patient's ears are exposed for both observation thereof and communication with the patient.

In the depicted embodiments of the invention, Velcro® has been used because of its adjustability and securing strength. Other fastening means or materials might readily be used, however, the simplicity and availability of Velcro® makes it the preferable fastening means. In like manner, the sleeve has been depicted as comprising bubble wrap, which is cheap and readily available. Other materials that perform the cushioning and waterproof functions of the sleeve as set forth in the foregoing for bubble wrap, might be used instead of bubble wrap, provided that it is also reasonably inexpensive so as to allow discarding thereof after a single use.

The patient restraint system of the invention, as set forth in the foregoing, is simple, cheap, uses mostly waterproof materials, all of which are commercially available, while insuring more than adequate stabilization of a patient's head and neck on a spine board. The system permits the patient to breath easily, to be aware (if conscious) of his or her surroundings, and to communicate with the emergency personnel.

It is to be understood that the various features of the present invention might be incorporated into other types of patient restraint systems, and that other modifications or adaptations might occur to workers in the art. All such variations or modifications are intended to be included herein as being within the scope of the present invention as set forth. Further, in the claims hereinafter, the corresponding structures, materials, acts and equivalents of all means or step-plus-function elements are intended to include any structure, material, or sets for performing the functions in combination with other elements as specifically claimed.

1. A head restraint system for use on a patient carrying member comprising:
a sleeve member having an open end and a substantially closed end; said open end being adapted to fit over one end of the patient carrying member;
said sleeve member having an exterior surface and a hollow interior, and being made of a cushioning material;
first and second side head restraint members being removably mountable on the exterior surface of said sleeve member; and
a vertical head restraint member being adapted to extend between said side head restraint members and to bear against a patient's head for holding it against the cushioning material of said sleeve.

2. A head restraint system as claimed in claim 1 wherein the exterior surface of said sleeve member is substantially smooth and the interior thereof has a cushioning surface.

3. A head restraint system as claimed in claim 2 wherein said sleeve member is made of bubble wrap material.

4. A head restraint system as claimed in claim 1 wherein a patch member is affixed to a portion of the exterior surface of said sleeve member.

5. A head restraint system as claimed in claim 4 wherein said patch member is spaced from a second patch member affixed to a portion of the exterior surface of said sleeve member.

6. A head restraint system as claimed in claim 4 wherein said patch member has a top surface of adhesive type material.

7. A head restraint system as claimed in claim 6 wherein the adhesion type material has a multitude of minute loops.

8. A head restraint system as claimed in claim 6 wherein said side head restraint member comprise first and second spaced head restraint blocks each having a bottom surface of adhesion type material adapted to mate with the adhesion type material on said top surface of said patch member.

9. A head restraint system as claimed in claim 8 wherein said adhesion type material on the bottom surface of each of said first and second head restraint blocks has a multitude of minute hooks.

10. A head restraint system as claimed in claim 8 wherein each of said first and second head restraint blocks has a top surface having a notch therein.

11. A head restraint system as claimed in claim 8 wherein said vertical head restraint member comprises an elongated strap member having first and second ends and a central portion, each of said ends comprising an adhesion type material.

12. A head restraint system as claimed in claim 12 wherein the adhesion type material on said first end is adapted to mate with the adhesion type material on said second end.

13. A head restraint system as claimed in claim 11 wherein said central portion of said strap member has a cushioning pad thereon adapted to bear against a patient's head.

14. A head restraint system as claimed in claim 13 wherein said pad has an adhesion surface adapted to contact the patient's head.

15. A head restraint system as claimed in claim 1 wherein said sleeve member comprise an upper member comprising a cushioning material and a lower member comprising a cushioning material, each of said upper and lower members having first and second side ends;
said first side ends of said upper and lower members being joined together by a first strip, at least a portion of which is an elastic material; and
said second side ends of said upper and lower members being joined by a second strip.

16. A head restraint system as claimed in claim 15 wherein at least a portion of said second strip is an elastic material.

17. A head restraint system as claimed in claim 15 wherein said first strip comprise an elongated portion of adhesion material attached to said first side end of said upper member, an elongated portion of material attached to said adhesion material, and a second elongated portion of adhesion material attached to said expansible material and to said first side edge of said lower member.

18. A head restraint system as claimed in claim 16 wherein second strip comprises an elongated portion of adhesion material attached to said second side end of said upper member, an elongated portion of expansible material attached to said adhesion material, and a second elongated portion of adhesion material attached to said expansible material and to said second side edge of said lower member.

19. A head restraint system for immobilizing a patient's head on a spine board comprising:
a hollow sleeve member fitted over the head end of the spine board, said sleeve member having a cushioning interior surface and a substantially smooth exterior surface;
said sleeve member having a top surface having a patch of hook-and-loop type adhesion material thereon;
first and second side head restraints removably affixed to said patch, each having a bottom surface having hook-and-loop type adhesion material thereon that mates with the hook-and-loop type material on said patch; and
a vertical head restraint member comprising an elongated strap having a first and second ends, each having thereon a hook-and-loop type material for mounting said strap to said sleeve, said strap having a central section having a head cushioning member mounted thereon.

20. A method of immobilizing the head and neck of a patient strapped to a carrying member, said method comprising:
fitting a cushioning sleeve over an end of the carrying member prior to strapping the patient thereto;
strapping the patient to the carrying member;
affixing first and second side head restraint block on either side of the patient's head to prevent sideways and rotational movement of the head;
placing a vertical restraint strap over the patient's forehead and mounting it on the sleeve on the carrying member.
